(19) United States
(12) Patent Application Publication
(10) Pub. No.: US 2022/0183805 A1
(43) Pub. Date: Jun. 16, 2022

(54) Dental Attachment System

(71) Applicant: Ivoclar Vivadent AG, Schaan (LI)

(72) Inventors: Alexander Grosse-Honebrink, Arbon (CH); Frank Müller, Feldkirch (AT); Andreas Schülke, Grabs (CH); Theresa Sujata Maria Senti, Triesenberg (LI)

(21) Appl. No.: 17/541,643

(22) Filed: Dec. 3, 2021

(30) Foreign Application Priority Data
Dec. 16, 2020 (EP) 20214669.2

(51) Int. Cl. A61C 13/235; A61C 7/00; A61C 7/14

(52) U.S. Cl. CPC A61C 13/235; A61C 7/14; A61C 7/006

(57) ABSTRACT

The present invention relates to a dental attachment system (100), having a first magnetic element (103-1) for fixing in an oral cavity (105); and a dental object (101) having a second magnetic element (103-2) for attachment to the first magnetic element (103-1).

### CROSS-REFERENCE TO RELATED APPLICATIONS

This application claims priority to European Patent Application No. 20214669.2 filed on Dec. 16, 2020, the disclosure of which is incorporated herein by reference in its entirety.

### TECHNICAL FIELD

The present invention relates to a dental attachment system, a dental object, and a method for attaching a dental object.

### BACKGROUND

Today's intraoral fixation options are realized, for example, by mechanical clamps or by means of a chemical adhesion. Clamping methods use, for example, clamps that are placed around one or more teeth, dental aligners that encompass a whole or part of an arch, or bands that are placed around one or more teeth. However, clamping procedures can damage tooth structure or displace teeth due to the pressure applied.

Adhesion is achieved by means of an adhesive or cement. However, attachment by means of an adhesive is permanent and must be removed by a dentist. This can cause problems for patients, as they cannot remove an attached dental object themselves during oral hygiene. An adhesive cream, on the other hand, only provides a weak attachment, so that it is not possible to wear a swallowable dental object overnight.

U.S. Pat. Nos. 4,209,905, 5,337,033, 4,508,507, 6,171,107, US007/0005042, and US2003/0118969 are directed to ways to attach elements to an oral cavity and are hereby incorporated by reference in their entirety.

### SUMMARY

It is the technical aim of the present invention to provide a releasable and reversible attachment of a dental object in the intraoral space.

This problem is solved by subject-matter according to the independent claims. Technically advantageous embodiments are the subject of the dependent claims, the description, and the drawings.

According to a first aspect, the technical problem is solved by a dental attachment system comprising a first magnetic element for fixing in an oral cavity; and a dental object comprising a second magnetic element for attachment to the first magnetic element. For example, the first magnetic element may be attached to one or more teeth, a brace, a bite splint, an abutment, a tooth stump, a denture, or a partial denture.

By means of a detachable magnetic holder through the two magnetic elements, the dental object can be removed from the tooth at any time. However, the magnetic element on the tooth can also remain in the mouth until it is removed by the dentist or the patient. Since the first magnetic element is applied to the previously cleaned tooth enamel, there is no problem with oral hygiene. The attachment system therefore has the technical advantage that non-prosthetic or non-orthodontic dental objects can be reversibly fixed in the intraoral region.

In a technically advantageous embodiment of the attachment system, the first magnetic element is formed by a magnetic varnish, a magnetic adhesive or a magnetic cement which can be applied to a tooth or teeth. This has, for example, the technical advantage that the first magnetic element can be arranged on any tooth in a quick and simple manner.

In a further technically advantageous embodiment of the attachment system, the magnetic coating comprises ferrimagnetic, ferromagnetic or permanent magnetic particles. The particles may have a size in the nanometer range or micrometer range. This has, for example, the technical advantage that a strong attractive force can be achieved between the magnetic elements.

In a further technically advantageous embodiment of the attachment system, the first magnetic element is formed by a permanent magnet or a ferromagnet which can be attached to the tooth or teeth. The attachment can be carried out by means of an attachment method of a varnish, an adhesive or a cement. This has the technical advantage, for example, of achieving a predetermined attractive force and good adhesion between the magnetic elements.

In a further technically advantageous embodiment of the attachment system, the first magnetic element is attached to a cap which can be placed on the tooth and/or can be attached or fastened to one or more teeth, a brace, a bite splint, an abutment, a tooth stump, a prosthesis, or a partial denture. The cap is, for example, a plastic cap that is adapted to the shape of the tooth and is placed on the tooth from above. This has the technical advantage, for example, that the magnetic element can be reversibly attached to the tooth in a simple manner.

In a further technically advantageous embodiment of the attachment system, the second magnetic element comprises a permanent magnet, an electromagnet and/or a ferromagnet. The second magnetic element may also be formed by a magnetic varnish. In this case, the first magnetic element can be a permanent magnet. This has, for example, the technical advantage that a good attractive force to the first magnetic element is achieved.

In a further technically advantageous embodiment of the attachment system, the second magnetic element or the dental object has a surface curvature that is adapted to a tooth. The surface curvature of the magnetic element or the dental object can thereby approximately follow the curvature of the tooth. This has the technical advantage, for example, that a displacement of the dental object relative to the tooth can be made additionally more difficult.

In a further technically advantageous embodiment of the attachment system, the second magnetic element comprises a layer that can be deformed by the attractive force between the two magnetic elements. The layer can be, for example, a foam layer or a sponge rubber layer that is depressed by the attractive force of the magnetic elements. This has the technical advantage, for example, of additionally stabilizing the dental object on the tooth.

In a further technically advantageous embodiment of the attachment system, the first magnetic element comprises a first contact surface to the second magnetic element and/or the second magnetic element comprises a second contact surface to the first magnetic element. The first and the second contact surfaces may be formed by flat surfaces, for example. This has, for example, the technical advantage that the distance between the tooth and the dental object is given by the contact surfaces and the dental object is stabilized so that wobbling of the dental object due to attraction between the two contact surfaces can be prevented.

In a further technically advantageous embodiment of the attachment system, spatial structures are formed in the first and second contact surfaces, which provide a predetermined orientation and/or a form fit between the first and second magnetic elements. The structures achieve, for example, the technical advantage that a rotation between the two magnetic elements and/or a displacement between the two magnetic elements can be prevented.

In a further technically advantageous embodiment of the attachment system, the dental object comprises a dental sensor, an electronic component, a battery and/or a housing. This has, for example, the technical advantage that physical properties on the tooth can be determined electronically.

In a further technically advantageous embodiment of the attachment system, the first magnetic element, the sensor and/or the sensor housing comprises one or more channels for guiding saliva to a sensor unit or control unit for the sensor. This also has, for example, the technical advantage that properties of the saliva can be analyzed by the dental object.

US 20080026344, 20210353180, 20210346690, 20210315460, and 20210282650 are directed to sensors for use in the oral cavity and are hereby incorporated by reference in their entirety.

In a further technically advantageous embodiment of the attachment system, the second magnetic element is arranged inside the dental object or on the outside of the dental object and/or the dental object comprises a housing that comprises a ferromagnetic, ferrimagnetic or permanent magnetic material or an electromagnet. This has, for example, the technical advantage that the magnetic element does not protrude to the outside and is protected from damage or moisture. In addition, for example, the technical advantage is achieved that the magnetic element is formed by and integrated in the housing. The housing may also comprise a battery or accumulator which is used for a power supply and also serves as a magnetic element.

According to a second aspect, the technical problem is solved by a dental object with a second magnetic element for attachment to a first magnetic element, which is attached in an oral cavity. The dental object solves the same technical advantages as the attachment system according to the first aspect.

According to a third aspect, the technical problem is solved by a method for attaching a dental object, comprising the steps of fixing a first magnetic element in an oral cavity; and attaching a dental object with a second magnetic element to the first magnetic element. The method solves the same technical advantages as the attachment system according to the first aspect.

### BRIEF DESCRIPTION OF THE DRAWINGS

Examples of embodiments of the invention are shown in the drawings and are described in more detail below.

It shows:

FIG. 1 a schematic view of a dental attachment system;

FIG. 2 a further schematic view of a dental attachment system;

FIG. 3 a structure for creating a form fit between the two magnetic elements;

FIG. 4 a schematic view of a magnetic element with a deformable layer;

FIG. 5 a schematic view of channels for guiding saliva; and

FIG. 6 a block diagram of the method for attaching a dental object.

### DETAILED DESCRIPTION

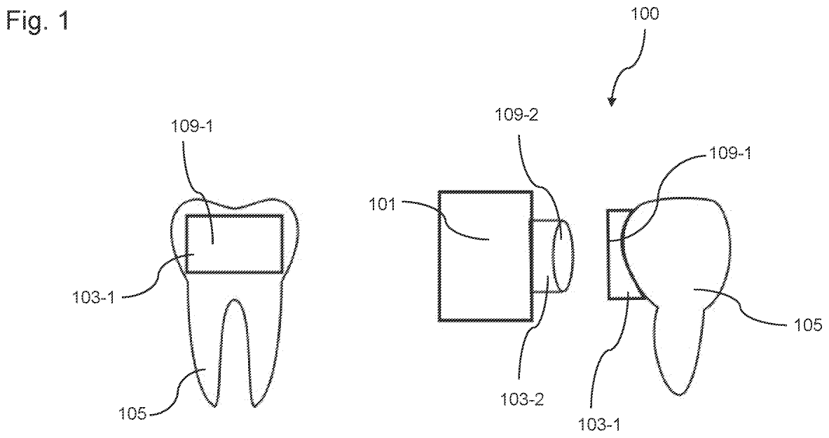

FIG. 1 shows a schematic view of a dental attachment system 100 for a dental object 101 in an oral cavity. The dental object 101 may be any spatial object that is to be secured in a patient's oral cavity. The dental object 101 has a geometry that allows it to be secured within the oral cavity. The dental object 101 may be attached to one or more teeth, a brace, a bite splint, an abutment, a tooth stump, a prosthesis, or a partial denture.

The first magnetic element 103-1 is a magnetic varnish that is manually applied to the tooth 105 in the selected intraoral region. This magnetic varnish includes, for example, health grade, polished and/or ferromagnetic or permanent magnetic particles having sizes in the nanometer or micrometer range mixed therein. For example, the magnetic varnish may comprise iron particles, neodymium particles (neodymium-iron-boron—NdFeB), or cobalt particles (aluminum-nickel-cobalt—AlNiCo) that serve to produce the magnetic properties of the magnetic element 103-1.

The magnetic varnish can be applied to the tooth 105. The magnetic varnish can be a pasty and/or light-curing material with magnetic fillers. This gives the advantage that the magnetic varnish can be applied flat or molded plastically. Molding can be carried out by means of a transparent casting template, for example made of silicone. The casting template can comprise a spatial structure that creates a spatial form-fit element in the cured magnetic lacquer, such as a triangular structure.

The magnetic varnish can, for example, be based on suitable polymers, such as copolymers of t-butyl acrylate, ethyl acrylate, methacrylic acid; copolymers of ethyl acrylate and methacrylic acid, copolymers of N-tert-butylacrylamide, ethylacrylate, acrylic acid; copolymers of vinyl acetate, crotonic acid and optionally other vinyl esters, anionic polysiloxanes, e.g. carboxy-functional, t-butyl acrylate, methacrylic acid. Examples of anionic polymers further include vinyl acetate/crotonic acid copolymers.

The first magnetic element 103-1 may also be a magnetic cement that is manually applied to the tooth 105 in the selected intraoral region. For example, the magnetic cement may be based on suitable materials, such as mono- or polyfunctional methacrylates, such as methyl, ethyl, 2-hydroxyethyl, butyl, benzyl, tetrahydrofurfuryl, or isobornyl (meth)acrylate, p-cumyl-phenoxyethylene glycol methacrylate (CMP-1E), bisphenol A dimethacrylate, Bis-GMA (an addition product of methacrylic acid and bisphenol adiglycidyl ether), ethoxy- or propoxylated bisphenol A dimethacrylate.

Magnetic adhesives can be: HEMA, Bis-GMA, D3MA, MDP, ethanol, water, methacrylate-modified polyacrylic acid, silica, camphorquinone, ethyl p-dimethylaminobenzoate, 2-dimethylaminoethyl methacrylate.

The dental object 101 includes a second magnetic element 103-2 that is used to attach to the first magnetic element 103-1. The magnetic element 103-2 of the dental object 101 acts as an opposite pole to the magnetic element 103-1 on the tooth 105, creating an attractive force between the two magnetic elements 103-1 and 103-2 that serves to secure the dental object 101.

Therefore, after applying the magnetic varnish to the enamel of the tooth 105, a dental object 101 equipped with the opposing magnetic element 103-2 can be attached to the modified tooth surface. For example, the magnetic element 103-2 of the dental object 101 may be formed by a permanent magnet (permanent magnet), a ferromagnet, or an electromagnet. The electromagnet of the dental object 100 may optionally be activated by electronics. The permanent magnet may be configured to generate a mechanically switchable magnetic field. For example, individual magnets of the permanent magnet may generate an effective magnetic field for attachment in a first spatial arrangement and cancel the magnetic field for attachment in a second arrangement. This effect arises from the superposition principle of the magnetic fields.

The magnetic element 103-2 may, for example, be formed by a ferromagnetic or permanent magnetic spatial body. For example, iron can be used as the ferromagnetic material. Neodymium, for example, may be used as the permanent magnetic material. This can be coated with various biocompatible materials such as gold. In general, however, any other magnetic material can be used by which a sufficient attractive force can be established.

The magnetic element 103-2 acts as a counter pole to the magnetic element 103-1 on the tooth 105. The ferromagnetic or permanent magnetic varnish on the tooth 105 also forms a counter pole to the magnetic element 103-2 inside the dental object 101 or on the dental object 101.

The dental object 101 to be attached may be, for example, a dental sensor, an electronic component, a battery, and/or a housing for various components. The dental sensor is a dental object 101 that can be arranged in the oral cavity and comprises electronics for autonomously detecting various measured values. For example, the dental sensor may comprise electronics for detecting a pH value in the oral cavity or for detecting other oral health issues.

The housing may be made of a polymeric material, metal, or a carbon fiber reinforced plastic (carbon material). In addition, the housing may include permanent magnetic or ferromagnetic particles such that the second magnetic element 103-2 is formed by and interspersed within the housing. In addition, it may be advantageous to arrange the second magnetic element 103-2 inside the housing, as it is then protected from corrosion. The dental object 101 has a size that can be arranged in the oral cavity of a patient. The housing can be produced in a three-dimensional printing process using a material with magnetic particles.

The magnetic varnish of the first magnetic element 103-1 can remain in the oral cavity until it is removed by the dentist or by the patient himself. However, since the magnetic varnish is only applied to the previously cleaned dental enamel, no oral hygiene problem arises after removal of the dental object 101.

Due to the magnetic fields exerted by the magnetic elements 103-1 and 103-2, the dental object 101 can be reversibly fixed in the oral cavity. In contrast to a clamping method, the magnetic varnish has no influence on the tooth position, so that no unwanted displacement of the tooth 105 takes place and due to the magnetic holder, the dental object 101 can be removed at any time.

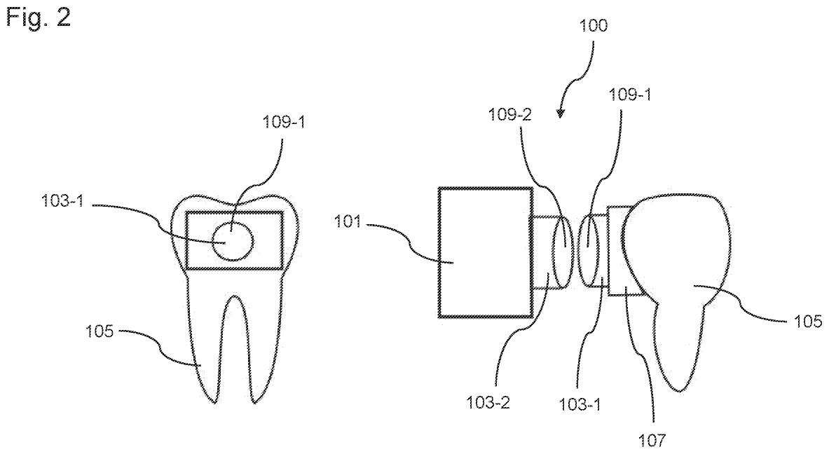

FIG. 2 shows a further schematic view of a dental attachment system 100. In this embodiment, a physical permanent magnet or ferromagnet is attached to the enamel of tooth 105 as the first magnetic element 103-1 by means of a dental adhesive, varnish, or dental cement.

Generally, the first magnetic element 103-1 may also be attached to the tooth 105 in other suitable ways. For example, the magnetic element 103-1 can also be attached to a cap that can be placed on the tooth 105. The cap is, for example, a plastic cap that is adapted to the shape of the tooth 105 and is placed on the tooth 105 from above. The cap allows the magnetic element to be reversibly attached to the tooth 105 in a simple manner.

This first magnetic element 103-1 also acts as an opposite pole to the second magnetic element 103-2 on the dental object 101, which is formed also by a physical permanent magnet or ferromagnet or an electromagnet.

In contrast to a clamping method, a cemented magnet as the first magnetic element 103-1 on the tooth 105 also has no influence on the tooth position and allows reversible removal of the dental object 101.

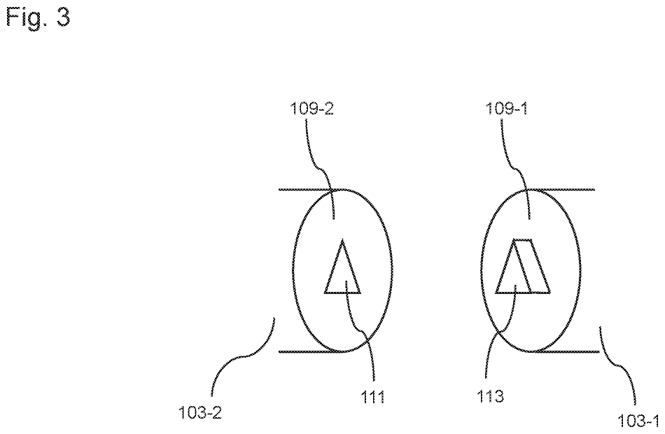

FIG. 3 shows a structure for establishing an orientation and a form closure between the two magnetic elements 103-1 and 103-2. The first magnetic element 103-1 comprises a first contact surface 109-1 to the second magnetic element 103-2 and the second magnetic element 103-2 comprises a second contact surface 109-2 to the first magnetic element 103-2. Spatial structures are formed in the first and second contact surfaces 109-1 and 109-2, respectively, which cause a form closure between the first and second magnetic elements 103-1, 103-2. This form closure allows the dental object 101 to be held in its intended position. Rotation and displacement of the dental object 101 can thus be prevented.

In this case, a recess 111 having a triangular profile is formed on the second contact surface 109-2. A corresponding protrusion 113 is formed in the opposite contact surface 109-1, which protrudes from the first contact surface 109-1. The protrusion 113 is held in the recess 111 by the attractive force between the two magnetic elements 109-1 and 109-2 and prevents mutual movement between the two magnetic elements 103-1 -and 103-2.

However, in general, other spatial interlocking structures may be used for orientation and positive engagement between the two magnetic elements 103-1 and 103-2.

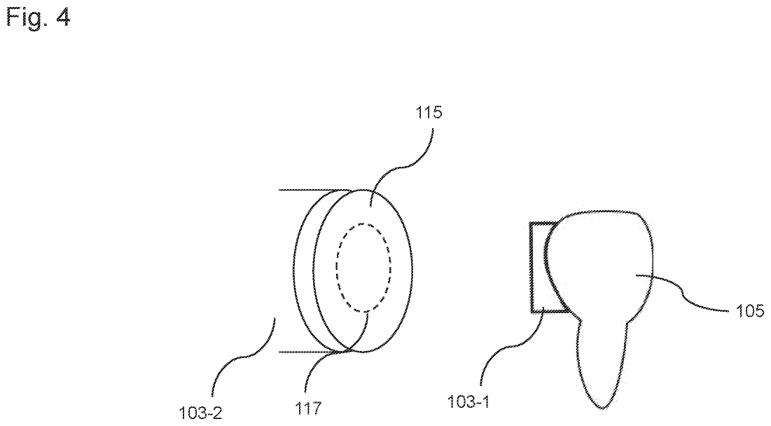

FIG. 4 shows a schematic view of a magnetic element 103-2 with a deformable layer 115. The layer is formed from a soft elastic material, such as a foam, a sponge rubber, or an elastomeric compound, such as silicone. Due to the attractive force between the two magnetic elements 103-1 and 103-2, the layer 115 is deformed in the area 117. This can prevent lateral displacement or mutual slippage of the magnetic elements 103-1 and 103-2.

Moreover, for this purpose, the magnetic element 103-2 or the dental object 101 may have a fixed surface curvature that corresponds at least approximately to the tooth 105 on which the dental object 101 is to be held. For example, the surface curvature may be created by a bulge in the contact surface 109-2 that corresponds to the shape of the tooth 105.

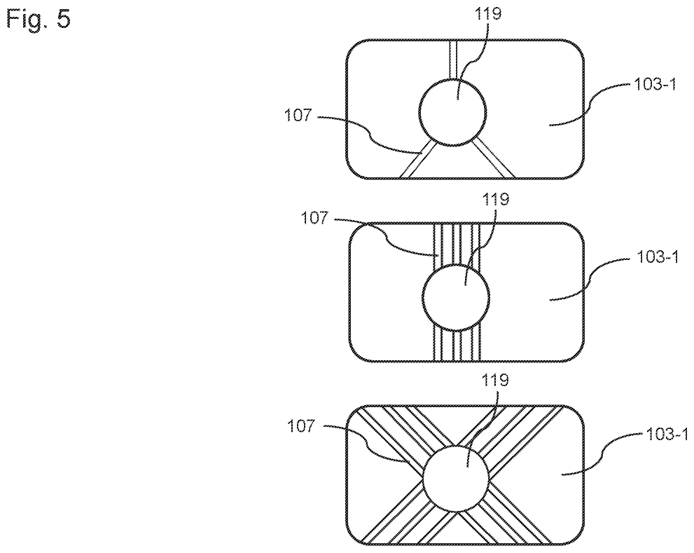

FIG. 5 shows a schematic view of channels for guiding saliva. The first magnetic element 103-1, the sensor, and/or the sensor housing may include one or more channels 107 for conducting saliva to a sensor unit. In this case, properties of the saliva may be analyzed through in the passageway 119. The channels 107 are for a fluid outflow or an air supply. The passageway opening 119 serves to enable measurement by a sensor unit and forms a measurement area for the sensor unit.

The channels 107 are formed in the adhesion area 103 by recesses and serve to supply liquid (saliva) to the sensor unit or to enable ventilation of the measuring area. The channels 107 can be arranged in a horizontal, diagonal, or vertical direction and can be formed in a U- or V-shape.

The salivary flow, in addition to the possibility of measurement, can also create a more comfortable wearing. In addition, the channels 107 cause material savings and easier removability. The channels 107 for saliva may be provided in the first magnetic element 103-1, the sensor and/or the sensor housing.

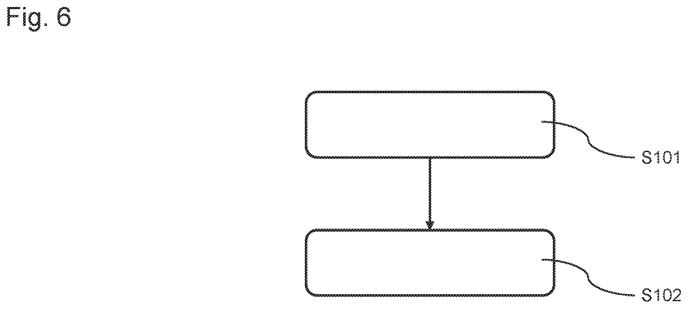

FIG. 6 shows a block diagram of the method for attaching the dental object 101. In a first step 5101, the first magnetic element 103-1 is attached to the tooth 105. In the second step S102, the dental object 101 is attached to the first magnetic element 103-1 by the second magnetic element 103-2.

The procedure uses magnetic fields to fix the dental object 101 in the oral cavity. These magnetic fields can generally be generated in different ways. Magnetic attachment using the two magnetic elements 103-1 and 103-2 can be significantly stronger in the shear direction and in the tensile direction than attachment using adhesive cream.

By means of a magnetic holder, the dental object 101 can be removed at any time. The magnetic varnish, magnetic adhesive, magnetic cement or the bonded or cemented permanent magnet can remain in the oral cavity until they are removed by the dentist or the patient. However, since the magnetic varnish, magnetic adhesive or magnetic cement are only applied to the previously cleaned enamel of tooth 105, no oral hygiene problem arises.

A strong, easily reversible attachment of the dental object 101 to the tooth 105 in the intraoral space is created. The reversibility is particularly advantageous, as a patient can perform his usual oral hygiene after manual removal of the dental object 101 and no secondary diseases are triggered by the dental object 101.

All features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by the features in question can be a method step of a method.

The scope of protection of the present invention is given by the claims and is not limited by the features explained in the description or shown in the figures.

### REFERENCE LIST

100 Attachment system

101 Dental object

103 Magnetic element

105 Tooth

107 Channel

109 Contact area

111 Recess

113 Lead

115 Shift

117 Area

119 Passage opening

1. A dental attachment system (100), comprising:
a first magnetic element (103-1) for fixing in an oral cavity; and
a dental object (101) having a second magnetic element (103-2) for attachment to the first magnetic element (103-1).

2. The dental attachment system (100) according to claim 1,
wherein the first magnetic element (103-1) comprises a magnetic varnish, a magnetic adhesive, or a magnetic cement that is applicable to a tooth (105) or teeth.

3. The dental attachment system (100) according to claim 2,
wherein the magnetic paint comprises ferrimagnetic, ferromagnetic, or permanent magnetic particles.

4. The dental attachment system (100) according to claim 1,
wherein the first magnetic element (103-1) comprises a permanent magnet or a ferromagnet attachable to the tooth (105) or teeth.

5. The dental attachment system (100) according to claim 1,
wherein the first magnetic element (103-1) is attached to a cap that is configured for placement on a tooth (105) or teeth and/or is configured for attachment to one or more teeth, a brace, a bite splint, an abutment, a tooth stump, a prosthesis, or a partial prosthesis.

6. The dental attachment system (100) according to claim 1,
wherein the second magnetic element (103-2) comprises a permanent magnet, an electromagnet and/or a ferromagnet.

7. The dental attachment system (100) according to claim 1,
wherein the second magnetic element (103-2) or the dental object (101) has a surface curvature adapted to a tooth (105).

8. The dental attachment system (100) according to claim 1,
wherein the second magnetic element (103-2) comprises a layer deformable by the attractive force between the two magnetic elements (103-1, 103-2).

9. The dental attachment system (100) according to claim 1,
wherein the first magnetic element (103-1) comprises a first contact surface (109-1) for contact to the second magnetic element (103-2) and/or the second magnetic element (103-2) comprises a second contact surface (109-2) for contact to the first magnetic element (103-1).

10. The dental attachment system (100) according to claim 9,
wherein spatial structures are formed in the first and second contact surfaces (109-1, 109-2) to provide a predetermined orientation and/or positive engagement between the first and second magnetic elements (103-1, 103-2).

11. The dental attachment system (100) according to claim 1,
wherein the dental object (101) comprises a dental sensor, an electronic component, a battery, and/or a sensor housing.

12. The dental attachment system (100) according to claim 11,
wherein the first magnetic element (103-1), the sensor and/or the sensor housing comprises one or more channels for guiding saliva to a sensor unit or a control unit.

13. The dental attachment system (100) according to claim 1,
wherein the second magnetic element (103-2) is arranged inside the dental object (101) or outside the dental object (101) and/or the dental object (101) comprises a housing comprising a ferromagnetic, ferrimagnetic or permanent magnetic material or an electromagnet.

14. A dental object (101) having a second magnetic element (103-2) for attachment to a first magnetic element (103-1) secured in an oral cavity (105).

15. A method of attaching a dental object (101), comprising the steps of:
fixing (S101) a first magnetic element (103-1) in an oral cavity (105); and
attaching (S102) a dental object (101) with a second magnetic element (103-2) to the first magnetic element (103-1).
